(19) United States
(12) Patent Application Publication
(10) Pub. No.: US 2017/0172623 A1
(43) Pub. Date: Jun. 22, 2017

(54) METHOD FOR ANKLE ARTHRODESIS

(71) Applicant: OLYMPUS CORPORATION, Tokyo (JP)

(72) Inventors: Ken FUJISAKI, Sagamihara-shi (JP); Takamitsu SAKAMOTO, Hachioji-shi (JP); Takeo USUI, Akiruno-shi (JP); Kenji MATSUMOTO, Hachioji-shi (JP)

(73) Assignee: OLYMPUS CORPORATION, Tokyo (JP)

(21) Appl. No.: 15/085,695

(22) Filed: Mar. 30, 2016

Related U.S. Application Data
(60) Provisional application No. 62/269,762, filed on Dec. 18, 2015.

(51) Int. Cl. A61B 17/56; A61B 17/86; A61B 17/32; A61B 17/16

(52) U.S. Cl. CPC A61B 17/56; A61B 17/1682; A61B 17/86; A61B 17/320068; A61B 2017/320056

(57) ABSTRACT

An ankle arthrodesis of this embodiment performs a cartilage the ultrasonic treatment procedure using an ultrasonic treatment tool. As one condition, when wear of cartilage has attained to the whole of an ankle, it carries out. A method for ankle arthrodesis is disposal which excises the cracked organization which exists in an ankle using the ultrasonic treatment tool, and fixes a tibia and a talus under it with a screw.

### CROSS-REFERENCE TO RELATED APPLICATIONS

This application is based upon and claim the benefit of priority from the prior U.S. Provisional Application No. 62/269,762 filed Dec. 18, 2015, the entire contents of which are incorporated herein by reference.

### BACKGROUND OF THE INVENTION

1. Field of the Invention

The present invention relates to a method for ankle arthrodesis using an ultrasonic treatment tool.

2. Description of the Related Art

In arthroscopic surgery, portals as small holes are generally made in a few locations around a joint (skin surface) and an arthroscope, a treatment tool and the like are inserted into these portals. Then, surgery is performed while videos in a monitor being checked in a state in which the inside of the joint is filled with a perfusion such as saline.

In arthroscopic surgery using such conventional treatment tools, there are causes for concern about some procedures. For example, if a drill or the like is used to make a bone hole, a hole is made only in a traveling direction of a drill blade and thus, when a bone hole is made inside a joint, an installation direction is limited by the position of a site to be treated. A treatment tool that cuts in a plane direction using a rotary blade cuts a bone by moving the rotary blade while rotating and thus, unevenness is left on the treatment surface and it is not easy to smooth the surface. Further, when a treatment tool using high frequencies is used, if thermal damage extending over surrounding tissues including the tissue to be treated is caused, it takes time before the postoperative condition becomes good.

### BRIEF SUMMARY OF THE INVENTION

A method for ankle arthrodesis in an embodiment according to the present invention includes: removing a cartilage of a talus using an ultrasonic vibration of an ultrasonic probe; creating a dimple using the ultrasonic probe in a region from which the cartilage of the talus has been removed; removing a cartilage of a tibia using an ultrasonic vibration of the ultrasonic probe; creating a dimple using the ultrasonic probe in a region from which the cartilage of the tibia has been removed; and fixing the talus and the tibia (using screws) while the region where the dimple of the talus is created and the region where the dimple of the tibia is created are in contact.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing a configuration example of an operation system including an ultrasonic treatment tool to perform ankle lateral ligament reconstruction according to an embodiment;

FIG. 2 is a diagram showing a state in which an arthroscope and a surgical instrument are inserted into an anterolateral inserting point and an anteromedial inserting point;

FIG. 3A is a diagram showing a configuration example of a probe provided with a treatment section having a rectangular opening;

FIG. 3B is a diagram showing a configuration example of the probe provided with the treatment section having a curette for ultrasonic vibration;

FIG. 3C is a diagram showing a configuration example of the probe provided with the treatment section having a plurality of steps of edges formed thereon;

FIG. 3D is a diagram showing a configuration example in which the tip of the treatment section of the probe has a circular shape;

FIG. 3E is a diagram showing a configuration example in which the tip of the treatment section of the probe has an elliptic shape;

FIG. 3F is a diagram showing a configuration example in which the tip of the treatment section of the probe has a long hole shape;

FIG. 3G is a diagram showing a configuration example in which the tip of the treatment section of the probe has a rectangular shape;

FIG. 4 is a diagram showing positions where an anterolateral inserting point and an anteromedial inserting point are created;

FIG. 5 is a diagram showing a state in which holes are formed in a subchondral bone plate by an ultrasonic treatment tool in which the tip of the treatment section of the probe is bent and has a needle shape;

FIG. 6 is a diagram illustrating a fixed state of the tibia and the talus by a method for ankle arthrodesis according to the present embodiment;

FIG. 7 is a diagram illustrating the fixed state of the tibia and the talus by a conventional method for ankle arthrodesis for comparison;

FIG. 8 is a diagram showing the fixed state of the tibia and the talus fixed by the method for ankle arthrodesis according to the present embodiment;

FIG. 9 is a diagram showing the fixed state of the tibia and the talus fixed by the conventional method for ankle arthrodesis for comparison; and

FIG. 10 is a flow chart illustrating surgical steps of the method for ankle arthrodesis.

### DETAILED DESCRIPTION OF THE PREFERRED EMBODIMENT

Hereinafter, a method for arthroscopic ankle arthrodesis using an ultrasonic treatment tool according to an embodiment of the present invention will be described with reference to the drawings.

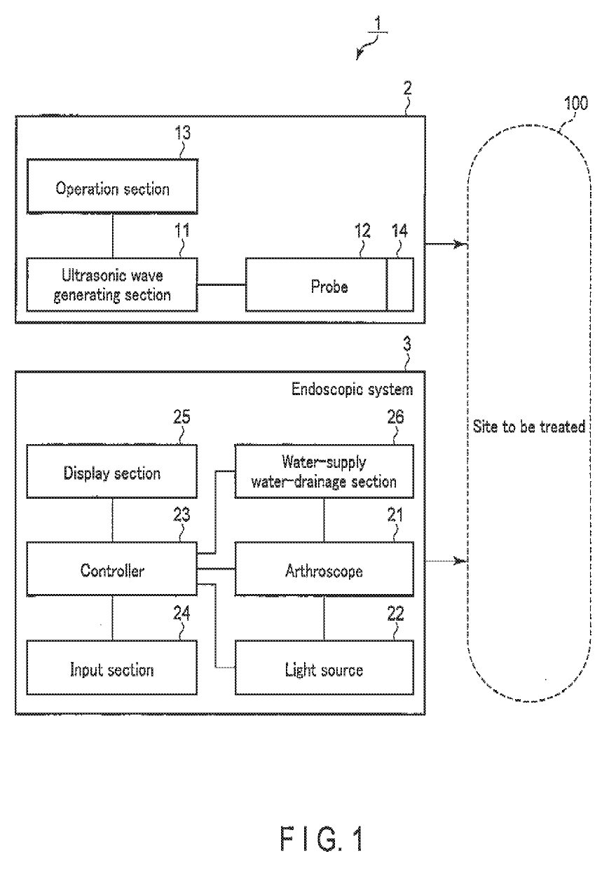

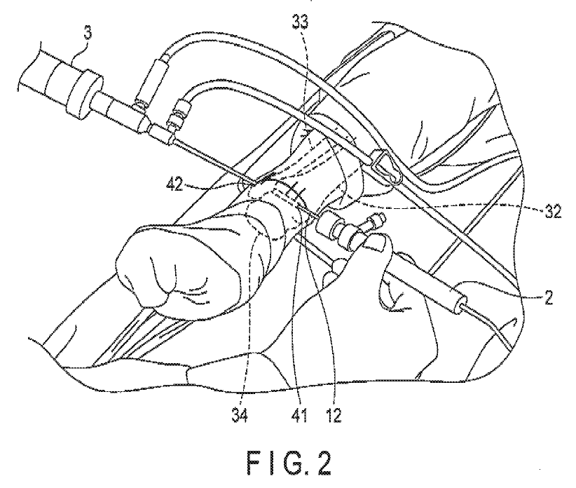

FIG. 1 shows a configuration example of an operation system including an ultrasonic treatment tool to perform a method for arthroscopic ankle arthrodesis according to the present embodiment. FIG. 2 is a diagram showing a state in which an arthroscope and an ultrasonic treatment tool are inserted into an anterolateral inserting point and an anteromedial inserting point. In the present embodiment described below, a site to be treated 100 will be described by taking an ankle as an example, but the present embodiment is not limited to the ankle and other joints can similarly be operated on easily using the ultrasonic treatment tool.

An operation system 1 according to the present embodiment includes an ultrasonic treatment tool 2 and an endoscopic system 3 including an arthroscope 21.

The ultrasonic treatment tool 2 includes an the ultrasonic wave generating section 11 that generates an ultrasonic vibration using an ultrasonic vibration element (for example, a piezoelectric element), a probe 12 that performs the ultrasonic treatment procedure of the site to be treated by transmitting the ultrasonic vibration, and an operation section 13 that performs an ON/OFF operation of the generation of an ultrasonic vibration by exercising drive control of the ultrasonic wave generating section 11.

The endoscopic system 3 includes the arthroscope 21 rigid endoscope, a visible light source 22 that radiates an illumination light of visible light as a light source of illumination light, a controller 23 that controls the endoscopic system 3 as a whole, an input section 24 such as a keyboard or a touch panel, a display section 25 that displays surgery information including imaged surgery conditions, and a water-supply/water-discharge section 26 that supplies physiological saline to the ankle lateral ligament of a site to be treated 100 and surroundings thereof or drains or perfuses physiological saline.

In the present embodiment, the water-supply/water-discharge section 26 is configured to supply physiological saline to the site to be treated through the arthroscope 21 or drain physiological saline, but a perfusion containing physiological saline or the like may be supplied or drained from the ultrasonic treatment tool 2.

As illustrated in FIG. 2, the probe 12 used in the present embodiment is an elongated rod-like member of about 2 mm to 4 mm in diameter extending linearly in a longitudinal direction and has a tip portion and a base end. The base end is linked to the ultrasonic wave generating section 11 and the tip portion is provided with a treatment section 14 that excises the cartilage.

The treatment section 14 of the probe 12 of the ultrasonic treatment tool 2 will be described.

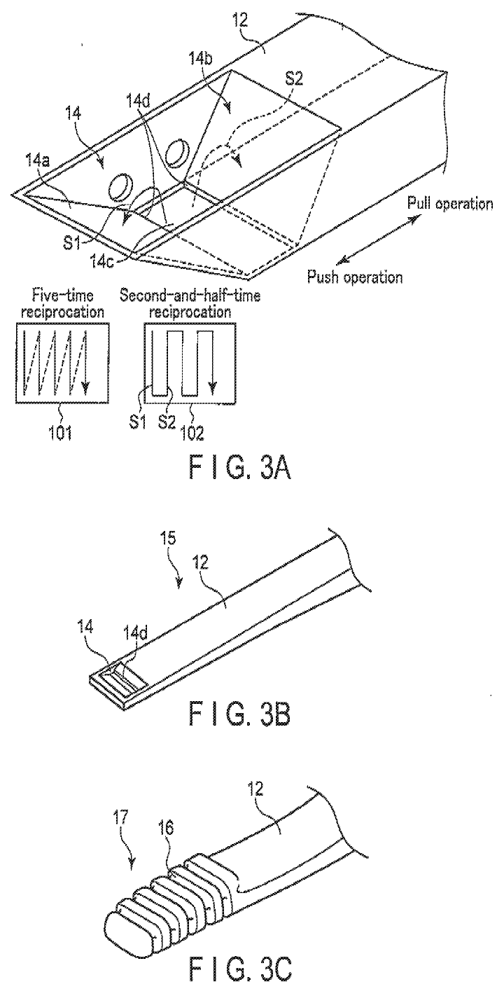

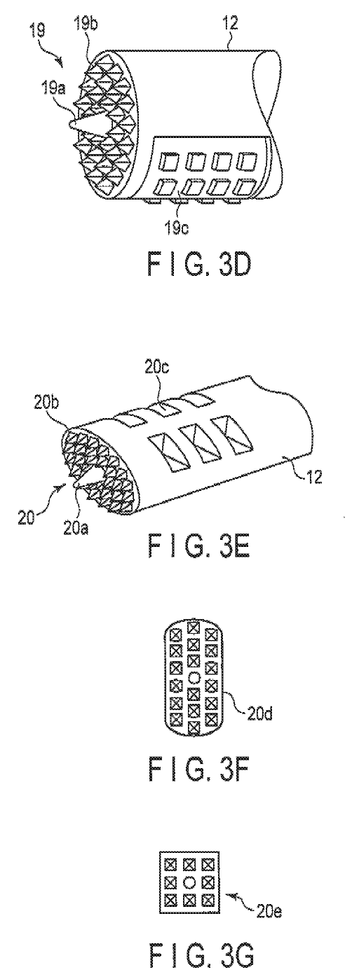

The treatment section 14 provided at the tip of the probe 12 illustrated in FIG. 3A has a shape in which an inverted isosceles trapezoid expands upward from below and an upper base of the trapezoid on the lower side is an opening 14c in a rectangular shape that cuts by being brought into contact with a site to be treated and a lower base of the trapezoid on the upper side is an opening to be an outlet of filing. Edges 14d are provided at respective ends of the opening 14c where inclined planes (legs) 14a, 14b inclined opposite to each other like expanding are in contact. These edges 14d function like a planer blade and can cut the site to be treated. Thus, the treatment section 14 of the probe 12 is structured to perform not only ultrasonic treatment S1 by a pull operation, but also an ultrasonic treatment S2 by a push operation by providing cutting edges forward and backward in a traveling direction of an opening that ablates the cartilage.

An edge is provided only on a tip side of an opening in a conventional curette and thus, after pulling nearer for the ultrasonic treatment, an operation to return to the original cutting start position is needed. To cut a surface 101, for example, a cutting operation of 5-time reciprocation is needed.

In contrast, the treatment section 14 according to the present embodiment can cut both ways and thus, when a surface 102 having the same area as the surface 101, a cutting operation of 2-and-half-time reciprocation is needed. That is, the ultrasonic treatment procedure of 5-time reciprocation is reduced to the ultrasonic treatment procedure of 2-and-half-time reciprocation for the same area and thus, the treatment section 14 according to the present embodiment has substantially 2-time cutting capacities when compared with the conventional curette and can make the dissection treatment more efficient.

FIG. 3B shows a configuration example in which the structure of the treatment section 14 in FIG. 3A is applied to an ultrasonic vibration curette 15 by providing the structure at the tip of the probe 12. If the curette 15 is produced with a thin blade of about 2 to 3 mm in thickness or by being bent, the cartilage and bone on a tibia surface and talus surface curved inside a joint fissure expanded to about 4 to 5 mm can be cut by being in traction in a state of ultrasonic vibration. The curette 15 may have elastically deforming characteristics to suitably adjust a pressing force.

FIG. 3C shows a treatment section 17 having a plurality of steps of edges 16 formed by a plurality of parallel grooves so as to rotate in a direction intersecting a longitudinal axis as a vibration direction of the probe 12. By providing edges on both sides of edges in the treatment section 17, the cartilage and bone are cut in both push and pull directions.

The probe 12 illustrated in FIG. 3D is provided with a treatment section 19 that opens a bone hole at the tip thereof and has protrusions 19c in a rectangular shape arranged checkerwise on the side face extending about halfway around the probe 12. The treatment section 19 has an apical protrusion 19a longer than other protrusions to align with a guide hole formed in advance arranged in the center of the tip surface and a plurality of protrusions 19b arranged therearound.

The treatment section 19 can carry out a cutting procedure of bones (cortical bones and cancellous bones) the cartilage and subchondral bone and all sites of living body tissues. That is, the cartilage can be shaved off by dissolution the cartilage using frictional heat generated between the treatment section 19 and the cartilage when the protrusions 19c made of edges formed on the side face of the probe 12 are put thereon. In addition, the bone is cut by being hammered by the protrusions 19b of the treatment section 19 provided at the tip of the probe 12 like ultrasonic vibration and extremely finely smashed and then shaved off. Therefore, by fitting the treatment section 19 at the tip of the probe to a shape matching an object to be cut, not only the type of the site to be cut, but also the amount of cutting and the shape thereof can appropriately be selected. Incidentally, though the amount of cutting is smaller than that of the protrusions 19b, the protrusions 19c provided on the side face of the probe 12 can knock and cut the bone.

A conventional drill used to form a bone hole opens a hole by rotation and thus, the basic shape of the hole is circular and even if the drill is moved in the left and right direction, the result is only an increased diameter in an indefinite shape and a desired shape cannot be formed. In contrast, the probe 12 of the ultrasonic treatment tool 2 does not rotate and moves infinitesimally in a forward and backward direction and thus, if the probe tip is hit against a bone and knocked vertically with respect to the bone surface, the bone hole is not limited to a circular shape and a bone hole in a shape matching the external shape of the treatment section (or a sectional shape of the probe) can be formed. By moving the location where the treatment section 19 is hit, a bone hole in any shape can be formed. If the shape of a bone hole to be formed is determined, for example, as illustrated in FIG. 3E, a bone hole in any shape can be formed by external appearance of a treatment section 20 provided with an apical protrusion 20a and a plurality of protrusions 20b at the tip of the probe 12 in an elliptic shape. In this example, grooves 20c are arranged so as to be arrayed in two rows in the longitudinal direction on the side face of the probe 12 to constitute edges for cutting. Similarly, a treatment section 20d in a long hole shape (track shape) illustrated in FIG. 3F or a treatment section 20e in a rectangular shape illustrated in FIG. 3G can form a bone hole in a shape matching the shape of the treatment section.

Further, the bone hole is not limited to a linear shape and a bone hole having a curved portion can also be formed. When, for example, a problem of being unable to fix a tendon or the like arises because only a short length (depth) of a bone hole can be taken depending on the shape of the bone if the bone hole is linear, fixing may be enabled by forming a curved bone hole. A bone hole having a curved portion can be implemented by using a probe having a curve in a tip portion.

Regarding the diameter or width of a bone hole, not only holes of the same diameter or the same width, but also holes in a tapering shape of the diameter or width from the inlet can be formed. In this case, the tapering shape can be formed with steps or without steps. If the probe tip having edges is pressed horizontally against the surface of a bone, vibrations occur like knocking while sliding on the treatment surface of a site to be treated and thus, the site can be excised by knocking and shaving off using edges. If the site to be treated has elasticity like the cartilage or fat, the site can be excised by shaving off using edges and fusing by frictional heat.

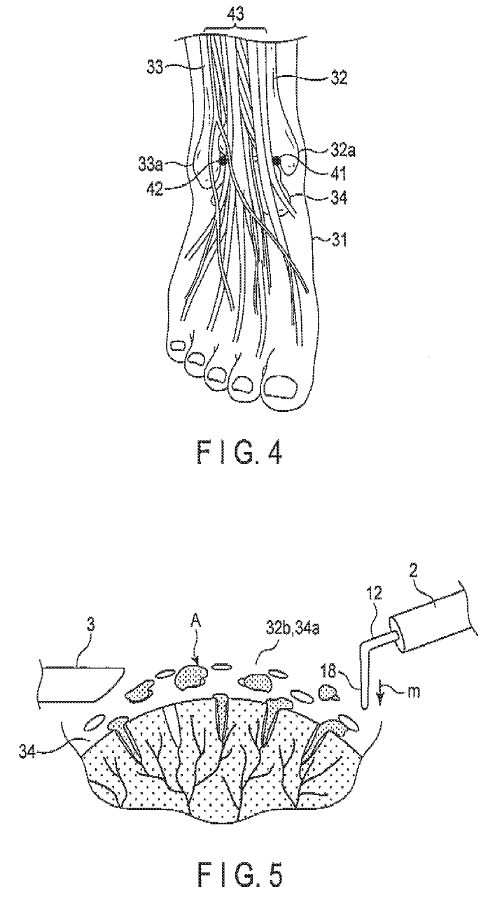

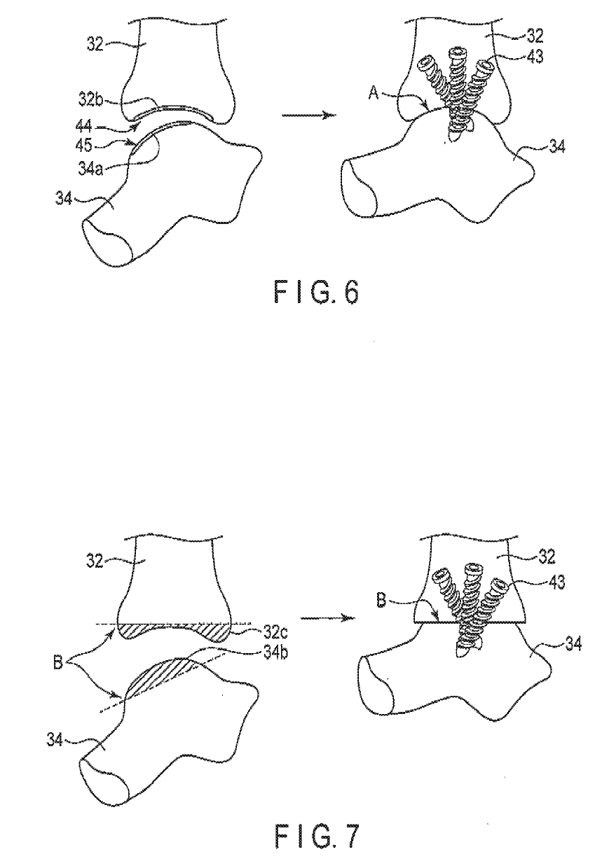

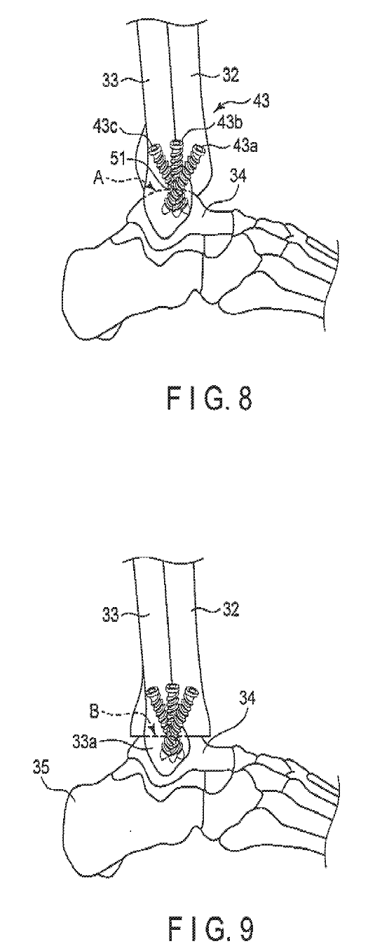

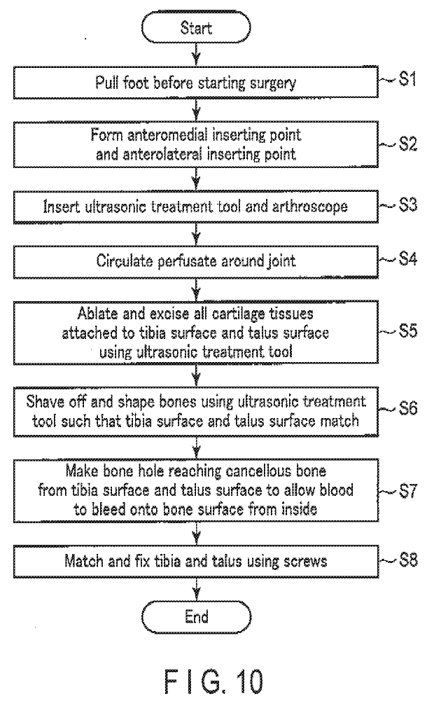

Next, the procedure of a method for arthroscopic ankle arthrodesis will be described with reference to FIGS. 2 to 10. FIG. 4 is a diagram showing an example of positions where an anterolateral inserting point and an anteromedial inserting point are created, FIG. 5 is a diagram showing a state in which holes are formed in a subchondral bone plate by an ultrasonic treatment tool whose tip has a needle shape, FIG. 6 is a diagram showing a fixed state of the tibia and the talus by a method for ankle arthrodesis according to the present embodiment, FIG. 7 is a diagram showing a conventional fixed state for comparison with FIG. 6, FIG. 8 is a diagram showing the fixed state of the tibia and the talus fixed by the method for ankle arthrodesis according to the present embodiment, and FIG. 9 is a diagram showing a conventional fixed state for comparison with FIG. 8. FIG. 10 is a flow chart illustrating surgical steps of the method for ankle arthrodesis.

The method for ankle arthrodesis according to the present embodiment is a method for ankle arthrodesis that forms a fusion surface of bone by successively removing a damaged cartilage and bone on the tibia surface and talus surface in an ankle using an ultrasonic treatment tool and fixing and fusing the tibia and the talus.

Before surgery is started, a leg is first placed on an operating table and a bandage is applied such that, as shown in FIG. 2, the instep falls to expand the joint fissure to be a space for treatment and the bandage is in traction by a tractor (not illustrated). For the traction, for example, a traction force of about 6 kg is applied for traction (step S1).

Then, an anteromedial inserting point 41 and an anterolateral inserting point 42 (portal) to insert the arthroscope 21 and the ultrasonic treatment tool 2 are formed (step S2). When determining positions thereof, as illustrated in FIG. 4, a large number of blood vessels/nerves, muscle-tendons and the like are arranged along a tibia 32, a peroneal bone 33, and a talus 34. Here, an anteromedial inserting point that is relatively safe is first created. The anteromedial inserting point 41 is formed in a position between a lower portion 32a of the tibia on the inner side and the talus 34 and the anterolateral inserting point 42 is formed in a position between the lower portion 32a of the tibia on the outer side, a lower portion of the peroneal bone 33, and the talus 34 by avoiding these vascular nerves, muscle-tendons and the like.

Next, as illustrated in FIG. 2, the ultrasonic treatment tool 2 is inserted from the anteromedial inserting point 41 and the arthroscope 21 is inserted from the anterolateral inserting point 42 (step S3). Incidentally, insertion locations may be interchanged in accordance with the location of the site to be treated. After the insertion, a perfusion made of physiological saline and the like is allowed to flow into the joint from the arthroscope 21 and the perfusion is circulated at a fixed flow rate by using a perfusion apparatus (not illustrated) (step S4).

A cartilage tissue 44 attached to a tibia surface 32b and a cartilage tissue 45 attached to a talus surface 34a illustrated in FIG. 6 are all excised by causing an ultrasonic vibration of the treatment section 14 of the probe 12 of the ultrasonic treatment tool 2 (step S5). If any osteophyte is found during cutting, the osteophyte is also excised. If any of the cartilage tissues 44, 45 and osteophyte remains between joint surfaces of the tibia surface and the talus surface while the joint is fixed by fusion, such a remnant could be an inhibition factor of fusion of the tibia and the talus.

Subsequently, curved surfaces are adjusted by the treatment section 14 provided at the tip of the probe so that curved shapes of the talus and the tibia are in close contact without any gap when the talus and the tibia are matched. That is, each bone is shaved off and shaped such that a concave curved surface of the tibia surface 32b and a convex curved surface of the talus surface 34a match by eliminating unevenness of each curved surface using the treatment section 14 vibrated ultrasonically to have curved surfaces of similar curves (step S6).

Next, the ultrasonic treatment tool 2 having the probe 12 including a treatment section 18 with an apical portion having a sharp tip as illustrated in FIG. 5 and bent halfway through the portion is used. Holes of a minimized area are made in 50 to 60 locations using the ultrasonic treatment tool 2 including the treatment section 18. The treatment section 18 has, as illustrated in FIG. 5, a bent tip and thus, instead of moving the ultrasonic treatment tool by applying a force in the longitudinal direction of the main body thereof, the ultrasonic treatment tool is moved in a direction perpendicular to the longitudinal direction and therefore, even if the joint fissure is narrow, drilling can easily be performed if only the tip portion of the treatment section 18 is inserted into the joint fissure.

A bone hole is formed in the tibia surface 32b and/or the talus surface 34a using the treatment section 18. The bone hole may be formed in, for example, a dimple shape of a golf ball. The bone hole is made so as to pierce through the subchondral bone plate and the bottom thereof reaches the cancellous bone to allow blood A to bleed onto the bone surface from inside (step S7). Incidentally, the bone hole is not limited to the dimple shape and any shape in which a small amount of blood remains on the surface may be adopted. This is because immobilization is promoted by Bone Marrow Stimulation contained in the bone marrow. The tibia 32 and the talus 34 are matched so that the tibia surface 32b and the talus surface 34a are brought into close contact as similar curved surfaces A.

Further, as illustrated in FIGS. 6 and 8, for example, a plurality of screws are used for screwing to retain a state in which the tibia 32 and the talus 34 are matched (step S8). After the aforementioned traction is removed, as illustrated in FIG. 6, a plurality of screws is screwed into the ankle under radiographic conditions if necessary. If, as a portion thereof, two or three screws are screwed in obliquely downward so as to intersect, for example, from outside the tibia, the fixing force until bones are fused is improved.

As a concrete procedure, first a guide wire is inserted from the outer surface of the tibia 32 toward the talus 34 in a direction to correct deformation.

Secondly, after the insertion of the guide wire into a body of talus is verified under radiographic conditions, a first screw 43a is screwed in by over-drilling.

Thirdly, a second screw 43b is screwed in from inside the tibia 32 into the body of talus. Here, care needs to be taken so that an intersection 51 of two screws is not placed in a high position of the talocrural articulation on both front and rear surfaces and side faces.

Fourthly, if sufficient space is available, a third screw 43c is screwed in. Then, a wound closure procedure is performed. This is done by suturing only the epidermis using a nylon yarn. At this point, care needs to be taken so that rami of superficial peroneal nerves running hypodermically are not sutured.

The method for ankle arthrodesis as described above is applied to cases in which activities of daily living (called ADL) are hindered due to pain derived from joints observed in degenerative ankle disease and articular rheumatism or deformation (irreversible). In the method for ankle arthrodesis, with an increasing range of motion of neighboring joint groups, the motion of the fixed ankle is compensated for.

Here, as a reference example for comparison with the present embodiment, a conventional representative method for ankle arthrodesis will be described. As illustrated in FIG. 7, a lower portion 32c of tibia in a concave shape of the tibia 32 and a lower portion 34b of talus in a convex shape of the talus 34 are cut along a plane B and these planes are matched and then, as described above, screwed using a plurality of screws 43.

The conventional method for ankle arthrodesis as described above is easier to perform surgically than the method in the present embodiment, but the tibia 32 and the talus 34 are partially cut relatively deeply and thus, when only one leg is operated on, the leg becomes shorter than the other leg that is not operated on, which makes it difficult for the patient to walk and poses a problem that the patient's upper body is tilted when walking.

Also in the present embodiment, cartilage tissues are excised and bones are formed by using an ultrasonic treatment tool. In contrast, when a conventional treatment tool is used to perform, like the present embodiment, the method for ankle arthrodesis that cuts cartilage tissues and adjusts curved shapes of bone, the procedure therefor is as described below:

First, all cartilage tissues are ablated using a curette or a rasp. Then, the bone is shaved using an ablator and further shaved beyond the subchondral bone plate in, for example, a dimple shape of a golf ball for bleeding. Next, the tibia 32 and the talus 34 are fixed by screwing using a plurality of screws.

If a conventional treatment tool is used for this surgery step, the following problems arise:

If a cartilage the ultrasonic treatment procedure is performed using a curette or a rasp, the treatment surface has a coarse finish. The tibia surface 32b and the talus surface 34a have a concave curve surface and a convex curved surface respectively and thus, a joint fissure expanded by traction is narrow and a treatment tool such as a curette and a rasp does not reach the rear side of the talar joint and a sufficient amount of the cartilage cannot be excised. Tissues of the remaining cartilage and the like could become, as described above, an inhibition factor of bone fusion. Further, in a rotating treatment tool such as an ablator and a drill, other tissues (anterior blood vessel/nerves) could be caught and the closest attention needs to be paid.

When a hole is made using a drill, Heat Necrosis may arise in bone due to frictional heat. In contrast, if an ultrasonic treatment tool is used, Heat Necrosis is minimized and bleeding to promote immobilization can be expected.

Further, when a bone hole for bleeding is formed using a drill, power is transmitted linearly and thus, no work can be done on the back side of the joint.

The present embodiment described above performs a cartilage the ultrasonic treatment procedure using an ultrasonic treatment tool and therefore, compared with the past, the following operation/working effects are achieved:
Due to micro-vibrations, the ultrasonic treatment tool can finely adjust the amount shaved off at a time by a pressing force of the treatment tool and also due to micro-vibrations, the treatment surface becomes smooth and an effect of promoting immobilization can be expected. Also, by producing the tip portion of the ultrasonic treatment tool thinly or in a thin blade shape, a region unreachable by conventional surgical instruments can be reached from the front portal. The tip portion of the ultrasonic treatment tool does not rotate and therefore, tissues are not caught during treatment.
As illustrated in FIG. 6, the cartilage is excised using an ultrasonic treatment tool and bone boring is performed on, for example, the subchondral bone. Compared with the conventional procedure, drilling using a drill may delay bone fusion due to Heat Necrosis in surrounding tissues. In contrast, if an ultrasonic treatment tool is used, Heat Necrosis and the bone architecture breakdown are minimized so that promotion of bone fusion at an early stage can be expected.
In the conventional procedure using an ablator, a driving force acts in the rotation direction due to rotation and therefore, it is difficult to create a smooth surface and the tip needs a certain thickness because of the need of a cover for the prevention of being caught. In the present embodiment, by contrast, a desired shape can be produced because the procedure proceeds in a direction in which a force is applied by devising a curved shape for the tip portion of the ultrasonic treatment tool.
When a conventional ablator is used, shaving scum of bone is large and it is difficult to suck, which could also lead to inflammation. In contrast, the ultrasonic treatment tool can finely shave only a surface in contact and therefore, shaving scum can be reduced and it is easy to suck.
The treatment sections of probes of the ultrasonic treatment tools illustrated in FIGS. 3A and 3B have a structure that ablates the cartilage also during a push operation, in addition to a pull operation, by providing edges (blades) for treatment forward and backward in a traveling direction of an opening that ablates the cartilage. With this structure, the ultrasonic treatment procedure of 5-time reciprocation is reduced to the ultrasonic treatment procedure of 2-and-half-time reciprocation for the same area.

A conventional current remove only when pulled and also a ultrasonic treatment tool in a conventional structure performs the ultrasonic treatment procedure only in any one direction, but the structure in the present embodiment performs the ultrasonic treatment procedure in both directions of the forward direction and the backward direction so that efficiency can further be improved.
In the past, if only the dimple shape is adopted for holes, the diameter increases when the depth is needed and therefore, an adhesive area of the talus and the tibia decreases and the extension of a bone fixing period is assumed. In addition, holes are normally formed in 50 to 60 locations and a lot of time is needed for boring.

In the present embodiment, by contrast, as illustrated in FIG. 5, an ultrasonic treatment tool whose tip has a needle shape is used for treatment and holes of a small area reaching the subchondral bone plate can be formed.

In the past, a treatment tool for treating the cartilage and a treatment tool for boring a hole are separate and a lot of work is needed, but an ultrasonic treatment tool in the present embodiment can treat both.
The ultrasonic treatment tool can be made as thin as possible and so, with its excellent accessibility, can easily treat a site unreachable by an existing treatment tool.
Ultrasonic vibrations of infinitesimal amplitude are used for shaving and therefore, the cut surface can be made smooth. Therefore, postoperative conditions are good. Further, ultrasonic vibrations are used for shaving and therefore, cutting is reliable, thermal damage to the site to be treated can be reduced, postoperative conditions are good, and minimal invasiveness is superior.
The ultrasonic treatment tool can treat both of soft tissues and hard tissues such as the cartilage and bone and therefore, replacement work of treatment tools can be reduced and burdens on engineers can be reduced.
Cutting is achieved by applying the treatment section vibrating ultrasonically and therefore, compared with a treatment section using a drill bit, almost no damage is done even if the treatment tool comes into contact with other sites that are not to be treated.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method for ankle arthrodesis comprising:
inserting an ultrasonic probe into a site to be treated positioned in a gap between a medial malleolus of a tibia and a talus in a direction from toes toward the tibia along the gap;
removing a soft tissue including a cartilage of the talus and the tibia using a treatment section provided at a tip of a probe vibrating ultrasonically of an ultrasonic treatment tool;
forming a bone hole reaching a cancellous bone in the talus from which the cartilage has been removed or at least one bone plane of the tibia using the treatment section vibrating ultrasonically to cause bleeding from the bone hole; and
fixing the talus and the tibia by inserting a plurality of screws from the tibia toward the talus while a region where a bone hole of the talus is formed and the tibia opposite thereto are matched or a region where a bone hole of the tibia is formed and the talus opposite thereto are matched.

2. The method for ankle arthrodesis according to claim 1, wherein, in the removing, when the talus and the tibia are matched, curved surfaces of the talus and the tibia are adjusted by the treatment section provided at the tip of the probe such that respective curved shapes of the talus and the tibia are in close contact without the gap.

3. The method for ankle arthrodesis according to claim 1, wherein, in the forming the bone hole, the ultrasonic treatment tool has an apical portion whose diameter is equal to the diameter of the bone hole formed by the treatment section and forms the bone hole reaching the cancellous bone in the talus from which the cartilage has been removed or at least the one bone plane of the tibia using the apical portion.

4. The method for ankle arthrodesis according to claim 3, wherein, in the forming the bone hole, the bone hole formed by the apical portion of the treatment section has a dimple in a hemispherical shape formed on a bone surface and the bone hole reaching the cancellous bone is formed in a center thereof.

5. The method for ankle arthrodesis according to claim 1, wherein, in the removing, the treatment section of the probe has an opening in a rectangular shape to ablate the cartilage, both sides of the opening perpendicular to a traveling direction during cutting are provided with edges for ablation, and the cartilage is ablated during reciprocation of the opening in both directions.

6. The method for ankle arthrodesis according to claim 1, wherein, in the forming the bone hole, the apical portion of the treatment section is provided with a dimple forming site in a hemispherical shape to form a dimple in a position of a length corresponding to a depth of the bone hole to be formed from the tip thereof.
